(19) United States
(12) Patent Application Publication
(10) Pub. No.: US 2007/0111929 A1
(43) Pub. Date: May 17, 2007

(54) Methods for the treatment of anxiety and for identification of anxiolytic agents

(75) Inventors: Chad Edward Beyer, Newtown, PA (US); Robert John Mark, Lawrenceville, NJ (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: Wyeth, Madison, NJ (US)

(21) Appl. No.: 11/508,441

(22) Filed: Aug. 23, 2006

Related U.S. Application Data
(60) Provisional application No. 60/710,385, filed on Aug. 23, 2005.

(51) Int. Cl. A61K 48/00; A61K 38/22; A61K 38/12

(52) U.S. Cl. 514/9; 514/12; 514/44

(57) ABSTRACT

Methods for the treatment of neuropsychiatric disorders such as anxiety are disclosed. The methods involve modulating the expression of the angiotensin IV receptor or modulating the biological activity of the angiotensin IV receptor by utilizing antagonists to the receptor. Also disclosed are methods for identifying antagonists of the angiotensin IV receptor that are effective to reduce anxiety in a subject.

### CROSS-REFERENCE TO RELATED APPLICATIONS

This Application claims benefit of U.S. Provisional Application No. 60/710,385 filed Aug. 23, 2005, which is hereby incorporated by reference in its entirety.

### FIELD OF THE INVENTION

The invention relates generally to the field of neuropharmacology. The invention features methods for the treatment of neuropsychiatric disorders such as anxiety. Also featured are methods to identify compounds that reduce anxiety in a subject.

### BACKGROUND OF THE INVENTION

Various publications, including patents, published applications, technical articles and scholarly articles are cited throughout the specification. Each of these cited publications is incorporated by reference herein in its entirety.

The angiotensin IV receptor (AT4R), also known as insulin-regulated membrane aminopeptidase (IRAP), was first described in 1992 as a high-affinity binding site for the hexapeptide angiotensin IV (AT4). (Swanson, GN et al. Regul. Pept. (1992) 40:409-19). The AT4R is a member of the MI family of zinc metallopeptidases and is a type II membrane-spanning protein, i.e., its active site is extracellular. (Keller, SR et al. J. Biol. Chem. (1995) 270:23612-18). Localization studies have demonstrated that AT4Rs are found in the kidney, heart, and adrenal tissue. (Baker, K M et al. Am. J. Physiol. (1990) 259:H324-32; Slinker, B K et al. Cardiovasc. Res. (1999) 42:660-9; Hamilton, T A et al. Peptides (2001) 22:935-44; and, Abrahamsen, C T et al. J. Pharmacol. Exp. Ther. (2002) 301:21-8). Within the central nervous system, western blot and in situ hybridization experiments showed that AT4R are found at high levels in the hippocampus and the entorhinal, prefrontal, and insular cortices. Levels in the substantia nigra, hypothalamus, and limbic areas, such as the amygdala, are also moderately high. (Thomas, W G et al. Int. J. Biochem. Cell Biol. (2003) 35:774-9). The differential distribution of AT4R in the brain has prompted considerable investigation into identifying a biological role for the receptors in central nervous system function.

Several literature reports indicate that AT4Rs influence various facets of cognitive function. For example, central infusions of one AT4R ligand, AT4, facilitate memory retention and retrieval in rodent passive avoidance paradigms. (Wright, J W et al. Brain Res. Bull. (1993) 32:497-502; and, Braszko, J J et al. Pharmacol. Res. (1998) 38:461-8). Similarly, chronic AT4 infusions were found to improve performance in the Morris Water Maze. (Pederson, E S et al. Regul, Pept. (1998) 74:97-103). Moreover, synthetic analogs of AT4 were found to reverse memory deficits induced by either scopolamine or bilateral perforant pathway lesion. (Perderson, E S (1998); and, Wright, J W et al. J. Neurosci. (1999) 19:3952-61). Consistent with the cognitive-enhancing role of AT4R, it has been reported that AT4Rs enhance both long term potentiation and potassium-evoked acetylcholine release in hippocampal slices. (Wayner, M J et al. Peptides (2001) 22:1403-14).

It is believed that the mechanism by which AT4 affects cognitive processes is by turning off the constitutively active peptidase activity of AT4R. Inhibition of AT4R activity results in elevated synaptic levels of several neuropeptides involved in cognitive processes Kovacs, G L and De Wied, D Pharmacol. Rev. (1994) 46:269-291). These neuropeptides include oxytocin, somatostatin, cholecystokinin 8, vasopressin, and substance P. (Herbst, J. J. et al. Am. J Physiol. (1997) 272, E600-E606; and, Matsumoto et al. Eur. J. Biochem. (2000) 267:46-52). While the exact recognition sequence for the peptidase activity has yet to be elucidated, blocking AT4R peptidase activity does not appear to affect other neuropeptides such as GnRH, neuropeptide Y, TRH, melanocortin, alpha-MSH, galanin, or calcitonin. Moreover, AT4 does not seem to inhibit AT4R by binding to the active site of the enzyme. Rather, AT4 binds to a juxtamembrane region to induce a conformational change in AT4R. The consequence of AT4 binding to AT4R is the inhibition of the peptidase activity of the AT4R. (Albiston, A L et al. Trends in Endo. Metabol. (2003) 43: 72-77).

Some reports suggest that oxytocin, one of the neuropeptides elevated as a result of AT4R repression, may exert an anxiolytic effect. Oxytocin knock-out mice showed higher levels of anxiety-related behavior when tested in the elevated plus maze test (EPM) for anxiety relative to wild-type mice. (Amico, J A et al. J. Neuroendocrinol. (2004) 16:319-24). In addition, central administration of synthetic oxytocin to oxytocin knock-out mice reduced anxiety levels as measured by EPM, and administration of an oxytocin receptor antagonist in addition to oxytocin in the knock-out mouse model abrogated the anxiolytic effects of the oxytocin. (Mantella, R C (2003)). Similarly, central administration of oxytocin to rats was found to attenuate the stress-induced neuroendocrine and molecular response in the brain. (Windle, R J et al. J. Neurosci. (2004) 24:2974-82).

In contrast, vasopressin, which is also elevated when AT4R is inhibited, is an anxiogenic neuropeptide. (Bhattacharya, S K et al. Biogenic Amines (1998) 14:367-86). Given the fact that the AT4R cleaves vasopressin more efficiently than it cleaves oxytocin (Lew, R A et al. J. Neurochem. (2003) 86:344-50), it seems that inhibition of AT4R in the central nervous system would be more likely to exert an anxiogenic, rather than an anxiolytic effect. However, studies reported heretofore have not addressed any relationship between AT4R activity and neuropsychiatric conditions such as anxiety.

Although most individuals experience feelings of anxiety within their lives, especially around new or important events, anxiety disorders are characterized by chronic and unremitting episodes of fear and nervousness that generally interfere with the individual's everyday life activities and experiences. Anxiety disorders are among the most common mental illness in the United States, affecting more than 19 million, or roughly 13% of adults between the ages of 18 and 54. (Source: U.S. National Institute of Mental Health). Anxiety disorders fall into several classes: Generalized Anxiety Disorder, characterized by constant, exaggerated worrisome thoughts about everyday routine life activities, and physical symptoms such as trembling, fatigue, insomnia, headaches, and nausea; Panic Disorders, characterized by repeated episodes of intense terror, and physical symptoms such as pounding heart, chest pains, lightheadedness, trembling, sweating, and hot flashes or chills; Phobias, characterized by disabling and irrational fears of specific objects or situations, which can lead to an individual avoiding such objects or situations unnecessarily; Obsessive Compulsive Disorder, characterized by repeated unwanted thoughts or compulsive behaviors that seem impossible to stop or control; and Post-Traumatic Stress Disorder, which generally occurs after witnessing or taking part in a terrifying event such as a rape, abuse, war, disaster, or serious accident, and physical symptoms such as insomnia, nightmares, flashbacks, depression, and irritability.

Anxiety disorders are typically treated with cognitive behavioral therapy and various medications. However, given the side effects of many drugs currently used to treat anxiety disorders, newer drugs and methods of treatment with fewer or less severe side effects are desirable. Moreover it is also desirable to obtain drugs that can work synergistically with existing therapies to enhance their efficacy, or that can target the underlying molecular, biochemical, or physiological basis for the anxiety disorder in question.

### SUMMARY OF THE INVENTION

The present invention describes methods for the treatment of neuropsychiatric disorders such as anxiety and methods to identify compounds for the treatment of neuropsychiatric disorders such as anxiety.

Some aspects of the invention feature methods for treating neuropsychiatric disorders in a subject in need of such treatment by administering to the subject a composition comprising a pharmaceutically acceptable carrier and an angiotensin IV receptor antagonist in an amount effective to diminish the biological activity of the AT4R. In a detailed embodiment, the neuropsychiatric disorder is anxiety. In a further detailed embodiment, the antagonist is angiotensin IV, divalinal-angiotensin IV, LVV-hemorphin 7, Nle-angiotensin IV, norleucinal-angiotensin IV, or any derivatives thereof.

The invention also features methods for treating neuropsychiatric disorders in a subject in need of such treatment by modulating the expression of the AT4R in the subject. In a detailed embodiment, the neuropsychiatric disorder is anxiety. In a further detailed embodiment, expression of the AT4R is reduced. In some embodiments, the expression of the AT4R on cell membranes is diminished. In some aspects, expression of the AT4R is modulated by an oligonucleotide that is antisense to a nucleic acid encoding the AT4R. In some embodiments expression of the AT4R is diminished by preventing localization of the AT4R to the cell surface by removing or altering the membrane translocation signal peptide, or by targeting the expressed AT4R for proteasome degradation.

The invention also provides methods for treating neuropsychiatric disorders in a subject in need of such treatment by blocking the active site of the AT4R with antibodies to the AT4R such that other molecules such as AT4R substrates cannot access the active site of the AT4R. In some embodiments, the neuropsychiatric disorder is anxiety.

Another aspect of the invention features methods for identifying antagonists of the AT4R. In some embodiments, the methods involve contacting a test compound with the AT4R and determining a decrease in the biological activity of the AT4R in the presence of the test compound relative to the biological activity of the AT4R in the absence of the test compound. In some embodiments, the method will utilize purified AT4R. In other embodiments, the method will be performed on a cell membrane comprising AT4R. In other embodiments, the method will be performed on whole cells expressing the AT4R. Compounds identified by this inventive method are also contemplated to be within the scope of the invention, as well as pharmaceutical compositions that comprise compounds identified by the inventive methods admixed with a pharmaceutically acceptable carrier.

Also provided are methods for identifying compounds that reduce anxiety in a subject by administering a test compound to the subject and determining a decrease in the level of anxiety in the subject relative to the level of anxiety in the subject in the absence of the test compound. Anxiety in a subject can be determined using such models as the four-plate model, elevated zero maze, elevated plus maze, light-dark transition test, Geller-type anticonflict test, Vogel-type anticonflict test, hole-board test, Morris water maze test, schedule-induced polydipsia model, stress-induced hyperthermia model, fear-potentiated startle model, maternal separation test, swim-despair test, or microdialysis. Compounds identified by this inventive method are also contemplated to be within the scope of the invention, as well as pharmaceutical compositions that comprise compounds identified by the inventive methods admixed with a pharmaceutically acceptable carrier.

The invention features methods for identifying compounds that reduce anxiety in a subject by contacting a test compound with the AT4R and determining a decrease in the biological activity of the AT4R in the presence of the test compound relative to the biological activity of the AT4R in the absence of the test compound, and then administering the test compound to a subject and determining a decrease in the level of anxiety in the subject relative to the level of anxiety in the subject in the absence of the test compound. Compounds identified by this inventive method are also contemplated to be within the scope of the invention, as well as pharmaceutical compositions that comprise compounds identified by the inventive methods admixed with a pharmaceutically acceptable carrier.

### BRIEF DESCRIPTION OF THE DRAWINGS

FIG. 1. Bar graph showing anxiolytic-like effect of AT4R blockade by AT4 in the mouse 4-plate model of anxiety. Acute AT4 administration produces anxiolytic-like effects in mice in a dose-dependent manner. Mice were administered systemic subcutaneous injections of vehicle or AT4 at 1, 3, and 10 mg/kg body weight (X axis), and evaluated in the mouse 4-plate model for anxiety, measuring number of punished crossings (Y axis). (*P<0.05 compared to vehicle.)

FIG. 2. Bar graph showing reversal of anxiolytic-like effects of AT4 administration by an oxytocin receptor antagonist. Mice were administered either vehicle, 3 mg/kg of AT4, 10 mg/kg of WAY-162720, an oxytocin receptor antagonist, or 3 mg/kg AT4 and 10 mg/kg of WAY-162720 (X axis), and evaluated in the mouse 4-plate model for anxiety, measuring number of punished crossings (Y axis). (*P<0.05 compared to vehicle.)

FIG. 3. Bar graph showing reversal of anxiolytic-like effects of AT4 administration by an AT4 receptor antagonist. Mice were administered either vehicle, 3 mg/kg of AT4, 5 nmol (icv) of divalinal, a AT4 receptor antagonist, or 3 mg/kg AT4 and 5 nmol (icv) of divalinal (X axis), and evaluated in the mouse 4-plate model for anxiety, measuring number of punished crossings (Y axis). (*P<0.05 compared to vehicle).

### DETAILED DESCRIPTION OF ILLUSTRATIVE EMBODIMENTS

As described herein, the inventors have demonstrated that inhibition of the AT4R produces anxiolytic effects in a widely used rodent model of anxiety that is predictive of effects in primates and humans. The anxiolytic effects observed by blocking AT4R activity parallel the effects observed by administering the anti-anxiety drug diazepam. Moreover, the anxiolytic effect has been shown by the inventors to be mediated through the neuropeptide oxytocin, inasmuch as those effects are reversed if the animal is co-administered an oxytocin antagonist.

Previous in vitro studies demonstrated that inhibition of the AT4R inhibited cleavage of both oxytocin and vasopressin. (Herbst (1997) and Matsumoto (2000)). Oxytocin is believed to be anxiolytic, but vasopressin is anxiogenic. (Bhattacharya, S K et al. Biogenic Amines (1998) 14:367-86). Because the AT4R cleaves vasopressin more efficiently than it cleaves oxytocin (Lew, R A et al. J. Neurochem. (2003) 86:344-50), it would have been expected prior to the present invention that inhibition of the AT4R protease activity would result in elevated synaptic levels of vasopressin, thereby inducing an anxiogenic effect, or at a minimum, offsetting any potential anxiolytic effects that may result from an increase in the level of oxytocin. The effects observed by the present inventors are contrary to these expectations.

The inventors' discovery that inhibition of the AT4R exerts an anxiolytic effect enables the practice of several methods in accordance with the present invention. These include methods of treating an individual for anxiety, as well as methods of identifying anxiolytic compounds that act through the AT4R pathway, as described in greater detail below.

Definitions

Various terms relating to the methods and other aspects of the present invention are used throughout the specification and claims. Such terms are to be given their ordinary meaning in the art unless otherwise indicated. Other specifically defined terms are to be construed in a manner consistent with the definition provided herein.

The term “treating” or “treatment” refers to any indicia of success in the attenuation or amelioration of a pathology or condition, including any objective or subjective parameter such as abatement, remission, or reduction of symptoms; increased tolerance by the subject to the pathology or condition; and improved physical or mental well-being of a subject. The indicia of success in the attenuation amelioration of a pathology or condition can be based on any objective or subjective parameters; including the results of a physical examination, neurological examination, and/or psychological or psychiatric evaluations.

The term “reduce anxiety” or “reducing anxiety” or “reduction of anxiety” refers to any measurable decrease, attenuation, or amelioration, including the elimination, of the symptoms of or the underlying psychological, molecular, biochemical, cellular, or physiological bases for anxiety.

“Effective amount” refers to an amount of a compound, material, or composition, as described herein effective to achieve a particular biological result. Such results may include, but are not limited to, treating neuropsychiatric disorders such as anxiety in a subject.

“In vivo” means within a living organism.

“In vitro” means within an artificial environment.

“Anxiety” refers to an emotional state comprising psychological, molecular, biochemical, cellular, and physiological responses to apprehension or fear of unreal or imagined danger. Anxiety includes, but is not limited to a generalized anxiety disorder, panic anxiety, obsessive compulsive disorder, social phobia, performance anxiety, post-traumatic stress disorder, acute stress reaction, adjustment disorders, hypochondriacal disorders, separation anxiety disorder, agoraphobia and specific phobias. Specific anxiety-related phobias which may be treated with the methods of the present invention are those commonly experienced in clinical practice including, but not limited to, fear of animals, insects, storms, driving, flying, heights or crossing bridges, closed or narrow spaces, water, blood or injury, as well as extreme fear of inoculations or other invasive medical or dental procedures.

“Anxiolytic” means any tendency to reduce anxiety.

“Anxiogenic” means any tendency to induce anxiety.

“Neuropeptide” means any molecule found in tissue from the peripheral or central nervous system comprised of at least two amino acids.

“Synapse” refers to the site of functional apposition between neurons, at which an impulse is transmitted from one neuron to another.

“Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

The term “AT4R antagonist” is used in the broadest sense, and includes any molecule that partially or fully blocks, inhibits, reduces, or neutralizes a biological activity of the angiotensin IV receptor.

“Biological activity” refers to any function or action of a molecule or ability to produce an effect in vitro or in vivo. With respect to the AT4R, such activity includes the protease/peptidase activity and all downstream effects thereof, including without limitation, anxiolytic or anxiogenic effects, signaling, glucose transport, enhancement of memory, reversal of amnesia, and the like.

As used herein, “test compound” refers to any purified molecule, substantially purified molecule, molecules that are one or more components of a mixture of compounds, or a mixture of a compound with any other material that can be analyzed using the methods of the present invention. Test compounds can be organic or inorganic chemicals, or biomolecules, and all fragments, analogs, homologs, conjugates, and derivatives thereof. Biomolecules include proteins, polypeptides, nucleic acids, lipids, polysaccharides, and all fragments, analogs, homologs, conjugates, and derivatives thereof. Test compounds can be of natural or synthetic origin, and can be isolated or purified from their naturally occurring sources, or can be synthesized de novo. Test compounds can be defined in terms of structure or composition, or can be undefined. The compound can be an isolated product of unknown structure, a mixture of several known products, or an undefined composition comprising one or more compounds. Examples of undefined compositions include cell and tissue extracts, growth medium in which prokaryotic, eukaryotic, and archaebacterial cells have been cultured, fermentation broths, protein expression libraries, and the like.

As used herein, “measure” or “determine” refers to any qualitative or quantitative determinations.

“Stable cell” or “stable cell line” refers to any cell in which any subunit of the AT4R or combinations thereof, including the whole AT4R, can be expressed so that antagonists of the AT4R can be identified and tested, and the roles of the AT4R in neuropsychiatric disorders such as anxiety can be examined.

“Antibodies” as used herein includes polyclonal and monoclonal antibodies, chimeric, single chain, and humanized antibodies, as well as antibody fragments (e.g., Fab, Fab', F(ab')₂ and Fᵥ), including the products of a Fab or other immunoglobulin expression library. With respect to antibodies, the term, “immunologically specific” or “specific” refers to antibodies that bind to one or more epitopes of a protein of interest, but which do not substantially recognize and bind other molecules in a sample containing a mixed population of antigenic biological molecules. Screening assays to determine binding specificity of an antibody are well known and routinely practiced in the art. For a comprehensive discussion of such assays, see Harlow et al. (Eds.), ANTIBODIES A LABORATORY MANUAL; Cold Spring Harbor Laboratory; Cold Spring Harbor, N.Y. (1988), Chapter 6.

Methods of Treatment

One aspect of the invention features methods for the treatment of neuropsychiatric disorders in a subject in need of such treatment. In some embodiments the method involves administering to the subject a composition comprising a pharmaceutically acceptable carrier and an angiotensin IV receptor antagonist in an amount effective to diminish the biological activity of the angiotensin IV receptor. In one preferred embodiment, the neuropsychiatric disorder is anxiety.

The AT4R antagonist can modulate the activity of the AT4R by inhibiting the active site of the AT4R, or by inducing a conformational change in the AT4R. The antagonist can be any organic or inorganic chemical, or biomolecule, or any fragment, analog, homolog, conjugate, or derivative thereof. Preferred examples of AT4R antagonists include, but are not limited to, angiotensin IV (Val-Tyr-Ile-His-Pro-Phe) (SEQ ID NO:1), Divalinal-Angiotensin IV, Nle-Angiotensin IV, Norleucinal Angiotensin IV, LVV-hemorphin-7 (Leu-Val-Val-Tyr-Pro-Trp-Thr-Gln-Arg-Phe) (SEQ ID NO:2), peptide analogs of LVV-hemorphin-7, including Leu-Val-Val-Tyr-Pro-Trp-Thr-Gln-Arg (SEQ ID NO:3), Val-Val-Tyr-Pro-Trp-Thr-Gln (SEQ ID NO:4), Val-Val-Tyr-Pro-Trp-Thr (SEQ ID NO:5), Val-Val-Tyr-Pro-Trp (SEQ ID NO:6), Val-Val-Tyr-Pro, Val-Val-Tyr (SEQ ID NO: 7), Val-Val-Tyr-Pro-Trp-Thr-Gln-Arg-Phe (SEQ ID NO:8), Val-Tyr-Pro-Trp-Thr-Gln-Arg-Phe (SEQ ID NO:9), Tyr-Pro-Trp-Thr-Gln-Arg-Phe (SEQ ID NO:10), Val-Tyr-Pro-Trp-Thr-Gln-Arg (SEQ ID NO:11), Val-Tyr-Pro-Trp-Thr-Gln (SEQ ID NO:12), Val-Tyr-Pro-Trp-Thr (SEQ ID NO:13), Val-Tyr-Pro-Trp (SEQ ID NO:14), Val-Tyr-Pro, Leu-Val-Val-Ala-Pro-Trp-Thr-Gln-Arg-Phe (SEQ ID NO: 15), Leu-Val-Val-Tyr-Ala-Trp-Thr-Gln-Arg-Phe (SEQ ID NO:16), Leu-Val-Val-Tyr-Pro-Ala-Thr-Gln-Arg-Phe (SEQ ID NO:17), Leu-Val-Val-Tyr-Pro-Trp-Ala-Gln-Arg-Phe (SEQ ID NO:18), Leu-Val-Val-Tyr-Pro-Trp-Thr-Gln (SEQ ID NO:19), Leu-Val-Val-Tyr-Pro-Trp-Thr (SEQ ID NO:20), Leu-Val-Val-Tyr-Pro-Trp (SEQ ID NO:21), Leu-Val-Val-Tyr-Pro (SEQ ID NO:22), or Leu-Val-Val-Tyr (SEQ ID NO:23). (Lee, J et al. J. Pharmacol. Exp. Therapeutics (2003) 305:205-11; and, Lew, R A. (2003)). Antibodies to the AT4R can also be used as antagonists. Such antibodies may be monoclonal or polyclonal, or may be in the form of an antisera.

The subject can be any animal, and preferably is a mammal such as a mouse, rat, hamster, guinea pig, rabbit, cat, dog, monkey, cow, horse, pig, and the like. Most preferably, the mammal is a human.

Preferred antagonists are those that provide a reduction in the peptidase activity of the AT4R of at least about 5%, and more preferably at least about 10%, at least about 15%, at least about 20%, at least about 25% at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or greater than 95% reduction in the peptidase activity of the AT4R, at a specified concentration of the antagonist. In one preferred embodiment, the reduction of peptidase activity of the AT4R modulates the concentration of anxiolytic or anxiogenic neuropeptides in the synapse. In a detailed embodiment, the synaptic concentration of anxiolytic neuropeptides are increased in the subject. In another detailed embodiment, the synaptic concentration of anxiogenic neuropeptides are decreased in the subject. In a more preferred embodiment, the synaptic concentration of anxiolytic neuropeptides are increased and the synaptic concentration of anxiogenic neuropeptides are decreased in the subject. Non-limiting examples of anxiolytic neuropeptides include oxytocin, galanin, and neuropeptide Y. Non-limiting examples of anxiogenic neuropeptides include vasopressin, somatostatin, corticotrophin releasing factor (CRF), and substance P.

The concentration of antagonist required to reduce the peptidase activity of the AT4R may vary with the species, breed, size, height, weight, age, overall health of the subject, the type of antagonist used, or the severity of the neuropsychiatric disorder. Determination of the proper concentration of antagonist required for a particular situation is within the skill of the art. In the inventive methods, the compositions comprise a concentration of antagonist in a range of about 0.001% to about 90% of the dry weight of the composition, or from about 1 pM to about 1 M. Dosage ranges may vary, e.g., from about 1 pg/kg body weight to about 1 g/kg body weight of the subject. A daily dose range of about 1 μg/kg to about 100 mg/kg of the weight of the subject is used in some embodiments, while a daily dosage range of at least about 0.01 mg/kg is used in other embodiments. Treatment can be initiated with smaller dosages that are less than the optimum dose of the antagonist, followed by an increase in dosage over the course of the treatment until the optimum effect under the circumstances is reached. If needed, the total daily dosage may be divided and administered in portions throughout the day.

The compositions can be prepared in a wide variety of dosage forms according to any means suitable in the art for preparing a given dosage form. Pharmaceutically acceptable carriers can be either solid or liquid. Non-limiting examples of solid form preparations include powders, tablets, pills, capsules, lozenges, cachets, suppositories, dispersible granules, and the like. A solid carrier can include one or more substances which may also act as diluents, flavoring agents, buffering agents, binders, preservatives, tablet disintegrating agents, or an encapsulating material. Suitable carriers are magnesium carbonate, magnesium stearate, talc, sugar, lactose, pectin, dextrin, starch, gelatin, tragacanth, methylcellulose, sodium carboxymethyl-cellulose, a low melting wax, cocoa butter, and the like. Non-limiting examples of liquid form preparations include solutions, suspensions, and emulsions, for example, water, alcohol, water propylene glycol solutions, and the like.

Administration of the compositions can be by infusion, injection (intravenously, intramuscularly, intracutaneously, subcutaneously, intraduodenally, intraperitoneally, and the like), intranasally, rectally, orally, or transdermally. Preferably, the compositions are administered orally.

For effective treatment of anxiety, one skilled in the art may recommend a dosage schedule and dosage amount adequate for the subject being treated. It may be preferred that dosing occur one to four times daily for as long as needed. The dosing may occur less frequently if the compositions are formulated in sustained delivery vehicles. The dosage schedule may also vary depending on the active drug concentration, which may depend on the needs of the subject.

Another aspect of the invention features methods for the treatment of neuropsychiatric disorders in a subject in need of such treatment by modulating the expression of the AT4R in the subject. In one preferred embodiment, the neuropsychiatric disorder is anxiety. In some embodiments, expression of the AT4R is modulated at the molecular level, for example, by diminishing the expression of the AT4R protein.

Expression of the AT4R may be specifically suppressed at the molecular level by utilizing antisense nucleic acids or RNA interference (RNAi). A review of RNAi is found in Marx, J. (2000) Science, 288:1370-1372. In brief, traditional methods of gene suppression, employing anti-sense RNA or DNA, operate by binding to the reverse sequence of a gene of interest such that binding interferes with subsequent cellular processes and blocks synthesis of the corresponding protein. Exemplary methods for controlling or modifying gene expression are provided in WO 99/49029, WO 99/53050 and WO 01/75164, the disclosures of which are hereby incorporated by reference in their entirety for all purposes. In these methods, post-transcriptional gene silencing is brought about by a sequence-specific RNA degradation process which results in the rapid degradation of transcripts of sequence-related genes. Studies have shown that double-stranded RNA may act as a mediator of sequence-specific gene silencing (see, for example, Montgomery and Fire, Trends in Genetics, 14:255-258, 1998). Gene constructs that produce transcripts with self-complementary regions are particularly efficient at gene silencing.

It has been demonstrated that one or more ribonucleases specifically bind to and cleave double-stranded RNA into short fragments. The ribonuclease(s) remains associated with these fragments, which in turn specifically bind to complementary mRNA, i.e., specifically bind to the transcribed mRNA strand for the gene of interest. The mRNA for the gene is also degraded by the ribonuclease(s) into short fragments, thereby obviating translation and expression of the gene. Additionally, an RNA polymerase may act to facilitate the synthesis of numerous copies of the short fragments, which exponentially increases the efficiency of the system. Gene-silencing may extend beyond the cell in which it is initiated such that the inhibition can result in biochemical, molecular, physiological, or phenotypic changes in other cells and systems throughout the organism.

Thus, available genetic information such as the nucleotide sequence, etc. of the AT4R can be used to generate gene silencing constructs and/or gene-specific self-complementary, double-stranded RNA sequences that can be delivered by conventional art-known methods. A gene construct may be employed to express the self-complementary RNA sequences. Alternatively, cells are contacted with gene-specific double-stranded RNA molecules, such that the RNA molecules are internalized into the cell cytoplasm to exert a gene silencing effect. The double-stranded RNA must have sufficient homology to the targeted gene to mediate RNAi without affecting expression of non-target genes. The double-stranded DNA is at least 20 nucleotides in length, and is preferably 21-23 nucleotides in length. Preferably, the double-stranded RNA corresponds specifically to a polynucleotide of the present invention. The use of small interfering RNA (siRNA) molecules of 21-23 nucleotides in length to suppress gene expression in mammalian cells is described in WO 01/75164. Tools for designing optimal inhibitory siRNAs include that available from DNAengine Inc. (Seattle, Wash.). See WO 01/68836. See also: Bernstein et al., RNA (2001) 7: 1509-1521; Bernstein et al., Nature (2001) 409:363-366; Billy et al., Proc. Nat'l Acad. Sci USA (2001) 98:14428-33; Caplan et al., Proc. Nat'l Acad. Sci USA (2001) 98:9742-7; Carthew et al., Curr. Opin. Cell Biol (2001) 13: 244-8; Elbashir et al., Nature (2001) 411: 494-498; Hammond et al., Science (2001) 293:1146-50; Hammond et al., Nat. Ref. Genet. (2001) 2:110-119; Hammond et al., Nature (2000) 404:293-296; McCaffrrey et al., Nature (2002): 418-38-39; and McCaffrey et al., Mol. Ther. (2002) 5:676-684; Paddison et al., Genes Dev. (2002) 16:948-958; Paddison et al., Proc. Nat'l Acad. Sci USA (2002) 99:1443-48; Sui et al., Proc. Nat'l Acad. Sci USA (2002) 99:5515-20. U.S. Patents of interest include U.S. Pat. Nos. 5,985,847 and 5,922,687. Also of interest is WO/11092. Additional references of interest include: Acsadi et al., New Biol. (January 1991) 3:71-81; Chang et al., J. Virol. (2001) 75:3469-3473; Hickman et al., Hum. Gen. Ther. (1994) 5:1477-1483; Liu et al., Gene Ther. (1999) 6:1258-1266; Wolff et al., Science (1990) 247: 1465-1468; and Zhang et al., Hum. Gene Ther. (1999) 10:1735-1737: and Zhang et al., Gene Ther. (1999) 7:1344-1349. These disclosures are herein incorporated by reference in their entirety for all purposes.

In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or MRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik et al., Proc. Natl. Acad. Sci. USA, 83:4143-4146 (1986)). The oligonucleotides can be modified to enhance their uptake, e.g., by substituting their negatively charged phosphodiester groups by uncharged groups.

There are a variety of techniques available for introducing nucleic acids into viable cells. The techniques vary depending upon whether the nucleic acid is transferred into cultured cells in vitro, ex vivo, or in vivo in the cells of the intended host. Techniques suitable for the transfer of nucleic acid into cells in vitro include the use of liposomes, electroporation, microinjection, cell fusion, DEAE-dextran, the calcium phosphate precipitation method, etc. The currently preferred in vivo gene transfer techniques include transfection with viral vectors and viral coat protein-liposome mediated transfection (Dzau et al., 1993, Trends in Biotechnology, 11:205-210). Viral vector mediated techniques may employ a variety of viruses in the construction of the construct for delivering the gene of interest. The type of viral vector used is dependent on a number of factors including immunogenicity and tissue tropism. Some non-limiting examples of viral vectors useful in gene therapy include retroviral vectors (see e.g., U.S. Pat. Nos. 6,312,682, 6,235,522, 5,672,510 and 5,952,225), adenoviral (Ad) vectors (see e.g., U.S. Pat. Nos. 6,482,616, 5,846,945 ) and adeno-associated virus (AAV) vectors (see, e.g., U.S. Pat. Nos. 6,566,119, 6,392,858, 6,468,524 and WO 99/61601). In some situations it is desirable to provide the nucleic acid source with an agent that targets the target cells, such as an antibody specific for a cell surface membrane protein or the target cell, a ligand for a receptor on the target cell, and the like. Where liposomes are employed, proteins which bind to a cell surface membrane protein associated with endocytosis can be used for targeting and/or to facilitate uptake, e.g., capsid proteins or fragments thereof tropic for a particular cell type, antibodies for proteins which undergo internalization in cycling, and proteins that target intracellular localization and enhance intracellular half-life. The technique of receptor-mediated endocytosis is described, for example, by Wu et al., J. Biol. Chem., 262:4429-4432 (1987); and Wagner et al., Proc. Natl. Acad. Sci. USA, 87:3410-3414 (1990). For review of the currently known gene marking and gene therapy protocols see Anderson et al., Science, 256:808-813 (1992).

Another aspect of the invention features methods for the treatment of neuropsychiatric disorders in a subject in need of such treatment by modulating the localization of the AT4R to the cell surface. In one preferred embodiment, the neuropsychiatric disorder is anxiety. In some embodiments, localization of the AT4R to the cell surface is modulated by targeting expressed AT4R for protease degradation. For example, ubiquitination of the AT4R can be utilized to target expressed AT4R to proteasomes. In some embodiments, localization of the AT4R to the cell surface is modulated by removing cell surface translocation signal peptides. Such signal peptides can be removed pre-transcriptionally or post-translationally.

Another aspect of the invention features methods for the treatment of neuropsychiatric disorders in a subject in need of such treatment by blocking the active site of the AT4R. By “blocking the active site” of the AT4R, it is meant that a chemical or biomolecule is utilized to obstruct the active site of the AT4R such that substrates of the AT4R cannot access the active site of the AT4R and thus are not cleaved by the AT4R. In one preferred embodiment, the neuropsychiatric disorder is anxiety. In some embodiments, the active site of the AT4R is blocked by antibodies to AT4R.

Methods for Screening Compounds

Another aspect of the invention features methods for identifying antagonists of the AT4R comprising contacting a test compound with the AT4R and determining a decrease in the biological activity of the AT4R in the presence of the test compound relative to the biological activity of the AT4R in the absence of the test compound.

For the screening assays, AT4R can be obtained from any source suitable in the art. The AT4R can be purified or bound to a cell membrane or membrane fragment. Purified AT4R, or subunits thereof, can be synthesized de novo, or obtained from any mammalian cell that naturally expresses the AT4R such as kidney, heart, adrenal, or brain tissue. Methods for purifying membrane-bound proteins are well established in the art, and commercial kits are also available such as the ProteoPrep Extraction Kit (Sigma, St. Louis, Mo.) and the Qprotome Cell Compartment Kit (Qiagen, Valencia, Calif.). Purified AT4R can also be obtained from the membranes of stable cells or cell lines that express the AT4R, such as transfected HEK 293T cells. (Lew, R A (2003)). Purified AT4R can also be obtained from recombinant expression systems, such as bacterial, yeast, insect cell systems, and the like. Screening assays can also be carried out on AT4R still bound to the cell membrane. Techniques of recombinant cloning and protein expression and purification are well established in the art.

Membrane-bound AT4R, or subunits thereof, can be obtained from any cell expressing the AT4R or subunits thereof. The cells can naturally express AT4R, such as mammalian kidney cells, cardiac cells, adrenal gland cells, or brain cells. The cells can be stable cells or stable cell lines induced to express AT4R such as transfected HEK 293T cells. (Lew, R A (2003)). Stable cells can be produced by any means suitable in the art for cloning and recombinant gene expression. Isolation of cell membranes or membrane fragments containing the AT4R can be carried out according to any means suitable in the art, including the membrane extraction method described by Mustafa et al. (Mustafa, T et al. J. Neurochem. (2001) 76:1679-87. In the alternative, whole cells whose membranes contain the AT4R can be used.

In one embodiment, interaction of a test compound with the AT4R is determined by any qualitative or quantitative technique known in the art. Determination of whether the test compound interacts with the AT4R can be carried out using binding assays wherein the test compound is labeled. The label can be any label suitable in the art such as radioisotopes, including ³H, ¹²⁵I, ³⁵S, ³³P, ³²P, ¹⁷⁷Lu, ⁹⁰Y, and the like; fluorophores, including FITC, phycoerythrin, rhodamine, Cy1, Cy2, Cy3, Cy4, Cy5, allophycocyanin, AlexaFluor® dyes (Invitrogen, Carlsbad, Calif.), fluorescent proteins, and the like; or enzyme labels, including phosphatase, luciferase, urease, peroxidase, oxidase, β-galactosidase, and the like. The binding assay can determine the equilibrium constant, dissociation constant, binding constant. Binding determinations can be made by any means suitable in the art, including without limitation, microscopy, equilibrium dialysis, ultrafiltration, spectroscopic analysis, chromatography, and calorimetry such as isothermal titration calorimetry. Competition assays may also be employed to determine the interaction with the test compound and the AT4R, such as those described by Mustafa et al. (Mustafa, T. (2001)), Lee et al. (Lee, J (2003)), and Lew et al. (Lew, R A (2003)).

The effect of the test compound on the biological activity of the AT4R can be determined by any means suitable in the art. The test compound can be assessed at multiple concentrations. A decrease in the biological activity of the AT4R can be measured in terms of a decrease in fluorescence resulting from cleavage of Leu-β-NA, a substrate of the AT4R, relative to the level of fluorescence observed in the absence of a test compound, or upon contacting the AT4R with a negative control compound. (Lew, R A (2003)). Alternatively, a decrease in the biological activity of the AT4R can be measured in terms of a decrease in cleavage of any other substrate of the AT4R. Such measurements can be carried out by any means suitable in the art, such as chromatography/HPLC, polyacrylamide gel electrophoresis, or mass spectroscopy. (Zhu, L, et al. J. Biol. Chem. (2003) 278:22418-23). Modulation of the biological activity of the AT4R can also be detertnined by measuring modulation of the concentration of neuropeptides that are known AT4R substrates. The modulation concentration of such neuropeptides can be measured in the synapse.

Another aspect of the invention features methods for identifying compounds that reduce anxiety in a subject by administering a test compound to the subject and determining a decrease in the level of anxiety in the subject relative to the level of anxiety in the subject in the absence of the test compound.

Baseline levels of anxiety and any reduction in anxiety resulting from the administration of the test compound to the subject can be measured using any means acceptable in the art. Such means may be with or without punishment to the subject. Non-limiting examples of assays used in the art for measuring anxiety include the Four-Plate Model, Elevated Zero Maze, Elevated Plus Maze, Light-Dark Transition Test, Geller-Type Anticonflict Test, Vogel-Type Anticonflict Test, Hole-Board Test, Morris Water Maze Test, Schedule-Induced Polydipsia Model, Stress-Induced Hyperthermia Model, Fear-Potentiated Startle Model, Maternal Separation Test, Swim-Despair Test, Microdialysis, and the like.

An additional aspect of the invention features methods for identifying compounds that reduce anxiety in a subject by a combination of an in vitro and in vivo screening assay. In one embodiment, a test compound is first screened in vitro to determine its physiologic, cellular, biochemical, or molecular effect, and then screened further in vivo to determine if the compound can reduce anxiety. In another embodiment, a test compound is first screened in vivo to determine if the compound can reduce anxiety, and then screened further in vitro to determine its physiologic, cellular, biochemical, or molecular effect.

In a detailed embodiment, the in vitro screening assay comprises identifying antagonists of the AT4R comprising contacting a test compound with the AT4R and determining a decrease in the biological activity of the AT4R in the presence of the test compound relative to the biological activity of the AT4R in the absence of the test compound. This embodiment can be practiced according to the details described herein. In a further detailed embodiment, the in vivo screening assay comprises identifying compounds that reduce anxiety in a subject comprising administering a test compound to the subject and determining a decrease in the level of anxiety in the subject relative to the level of anxiety in the subject in the absence of the test compound. This embodiment can be practiced according to the details described herein.

Compounds identified by any of the foregoing inventive screening methods are contemplated to be within the scope of this invention. Such compounds are preferably anxiolytic. Such compounds may be formulated as a pharmaceutical composition by admixing such compound in an amount effective to reduce anxiety in the subject to which it is administered and a pharmaceutically acceptable carrier, as described herein. Such pharmaceutical compositions can be administered to a subject according to the methods of the invention in order to treat anxiety in the subject.

The following examples are provided to illustrate the invention in greater detail. The examples are intended to illustrate, not to limit, the invention.

### EXAMPLE 1

### Effect of AT4 Receptor Blockade on Anxiety Behavior in Mouse 4-Plate Model

The effects of AT4 receptor blockade by AT4 were investigated in the mouse 4-plate model of anxiety.

Male Swiss Webster mice weighing 18-24 g were used in the 4 plate studies. Animals were housed in groups of 15 in an AAALAC-accredited facility (Wyeth Research, Princeton, N.J.) with food and water available ad libitum. Animals were maintained on a 12-hour light/dark cycle (lights on at 0600) with all studies performed during the light phase. On the day of experiments, mice were injected with AT4 (0, 1, 3 and 10 mg/kg) 30 minutes before the start of the study. Initially, mice were individually placed in a plexiglass cage (18×25×16 cm) with a floor consisting of four rectangular metal plates (8×11 cm), which are wired to a shock generator (Med Associates). In each experiment, mice were placed into the chamber and given an 18-sec habituation period, which was followed by a 1-min test session. After the habituation period, an electric shock (0.8 mA) was delivered for 3.0 sec when mice crossed from one plate to another. The crossing from one plate to the next is referred to as a “punished crossing.” The number of punished crossings during a 1-min test period was recorded by a computer. The mean number of punished crossings for each group was expressed as a percentage of the value observed in the control animals. Data were subjected to an overall one-way analysis of variance (ANOVA) and post-hoc comparisons were made by a contrast using least squares. Significant differences in treatment occurred when p<0.05 compared to vehicle.

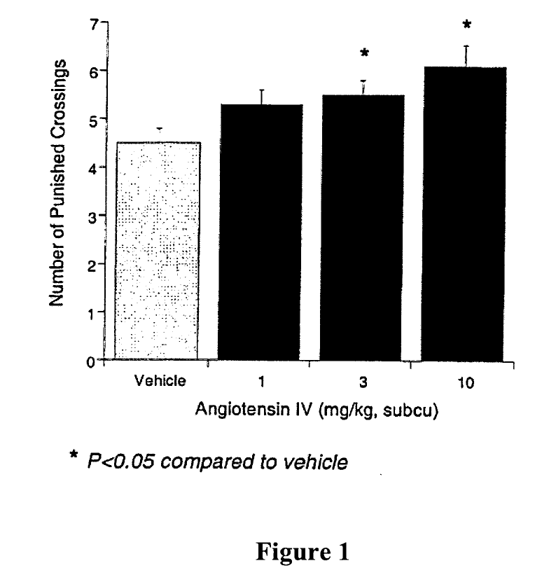

Results are shown in FIG. 1. As can be seen, acute treatment with 3 and 10 mg/kg of AT4 significantly (p<0.05) increased the number of punished crossing compared to animals treated with vehicle alone. The results are similar to those observed with known anti-anxiety drugs such as Valium (diazepam) in this model.

### EXAMPLE 2

### Reversal of Anxiolytic-Like Effects of AT4 by an Antagonist of Oxytocin

To determine whether the anxiolytic-like effects of AT4 Receptor Blockade were mediated, at least in part, by oxytocin, the procedures set forth in Example 1 were repeated in the presence of a known oxytocin receptor antagonist, WAY-162720.

For these studies, the same 4-plate procedures were used as described in Example 1, above. The only difference was that animals were injected with 10 mg/kg of the oxytocin receptor antagonist, WAY-162720. This injection was given at the same time as AT4 (3 mg/kg) which was administered 30 minutes before mice were placed in the 4-plate cage. After the 18 sec habituation period, an electric shock (0.8 mA) was delivered for 3.0 sec when mice crossed from one plate to another. A 3-sec time followed the delivery of each shock and a computer recorded the number of punished crossings during a 1-min test period. The mean number of punished crossings for each group was expressed as a percentage of the value observed in the control animals. Data were subjected to an overall one-way analysis of variance (ANOVA) and post-hoc comparisons were made by a contrast using least squares. Significant differences in treatment occurred when p<0.05 compared to vehicle.

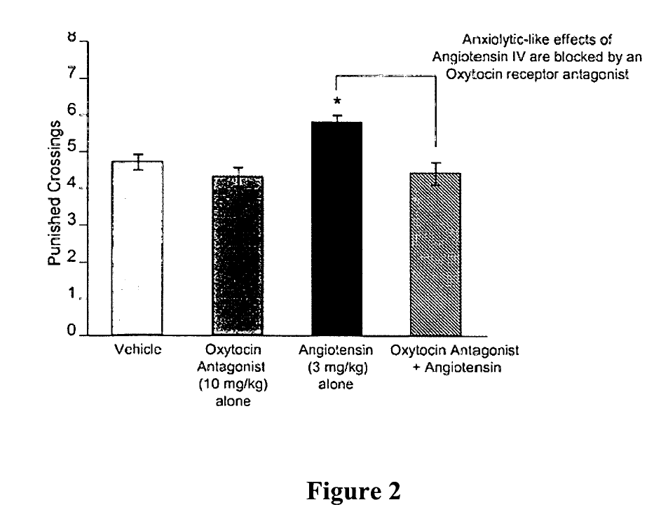

Results are shown in FIG. 2. As can be seen, acute treatment with WAY-162720 produced no effect on behavior when tested alone, and acute treatment with AT4 increased the number of punished crossings compared to animals administered the vehicle control. Acute treatment with WAY-162720 and AT4 showed that this oxytocin receptor antagonist completely blocked the anxiolytic effects of AT4 in the 4-plate model.

### EXAMPLE 3

### Effect of AT4 Receptor Blockade on Oxytocin Levels in Rat Amygdala

In vitro, AT4 inhibits the peptidase activity of the AT4 receptor, leading to increases in levels of several peptides including oxytocin. To confirm this observation in vivo, microdialysis coupled to immunoassay techniques were used to monitor basal and AT4-induced changes in extracellular levels of oxytocin in the rat amygdala.

For microdialysis protocols, male Sprague-Dawley rats, weighing between 280 and 350 g, were group housed in an AAALC-accredited facility and maintained on a 12 hr light/dark cycle. All procedures were conducted during the light period (lights on at 0600 h). Using 2-3% halothane (Fluothane; Zeneca, Cheshire, UK) anesthesia, animals were secured in a stereotaxic frame with ear and incisor bars (David Kopf, Tujunga, Calif.). A microdialysis guide cannula (CMA/12; CMA Microdialysis, Stockholm, Sweden) was directed toward the rat amygdala using the following coordinates: A/P—2.7 mm M/L—4.6 mm and D/V—7.2 mm (Paxinos, G and Watson, C. The Rat Brain in Stereotaxic coordinates, 1986, Academic Press). Guide cannula was fixed to the skull with two stainless-steel screws (Small Parts, Roanoke, Va.) and dental acrylic (Plastics One, Roanoke, Va.). Following surgery, animals were individually housed in Plexiglass cages (45 cm sq.) for approximately 24 hours and had access to food and water ad libitum. Following a 24 hr post-operative recovery, a pre-washed microdialysis probe (CMA/12; OD 0.5 mm, membranes length 2 mm, 20 kD cut-off) was perfused with artificial CSF (aCSF; 125 mM NaCl, 3 mM KCI, 0.75 mM MgSO₄ and 1.2 mM CaCl₂, pH 7.4) at flow rate of 0.2 ml/min for at least 18 hours prior to experimentation. On the day of procedures, microdialysis probes were inserted, via the guide cannula, into the amygdala and perfused with aCSF at a flow rate of 1 μl/min. A 3-hour stabilization period was allowed following probe insertion before any neurochemical were measured. Thirty-minute samples were collected for 2 hours to establish a steady baseline. These samples were immediately placed on dry ice. Next, AT4 was infused directly thru the probe into the amydala for 60 minutes. Once the injection was complete, samples were collected for 3 hours post-infusion to evaluate a timecourse of AT4 effects. Following collection, all samples were stored on dry ice. Oxytocin levels from dialysis samples were quantified by an oxytocin immunoassay (cat no. DE1900; R&D Systems, Inc) according to conditions specified by the manufacturer.

Intra-amygdala infusion (60 min) of 1 and 10 uM Nle-AT4 resulted in a concentration-dependent increase in amygdala levels of oxytocin (83% and 128% above baseline, respectively). Additionally, a systemic injection of Nle-AT4 (0.5 mg/kg, s.c.) produced marked elevations in amygdala levels of oxytocin (5-fold) compared to vehicle-treated animals, suggesting that this peptide readily enters the central nervous system.

### EXAMPLE 4

### AT4 Receptor Antagonist, Divalinal, Blocks the Anxiolytic Properties of Angiotensin IV

To determine whether the AT4 receptor mediates the anxiolytic-like properties of AT4, the procedures set forth in Example 2 were repeated in the presence of the known AT4 receptor antagonist, divalinal.

For these studies, the same 4-plate procedures were used as described in Example 1, above. The only difference was that animals were injected intracerebroventricularly (icv) with 5 nmol of the AT4 receptor antagonist, divalinal. AT4 (3 mg/kg) and divalinal were administered 30 and 20 min, respectively, prior to being placed in the 4-plate cage to habituate. After the 18 sec habituation period, an electric shock (0.8 mA) was delivered for 3.0 sec when mice crossed from one plate to another. A computer recorded the number of punished crossings during a 1-min test period. The mean number of punished crossings for each group was expressed as a percentage of the value observed in the control animals. Data were subjected to an overall one-way analysis of variance (ANOVA) and post-hoc comparisons were made by a contrast using least squares. Significant differences in treatment occurred when p<0.05 compared to vehicle.

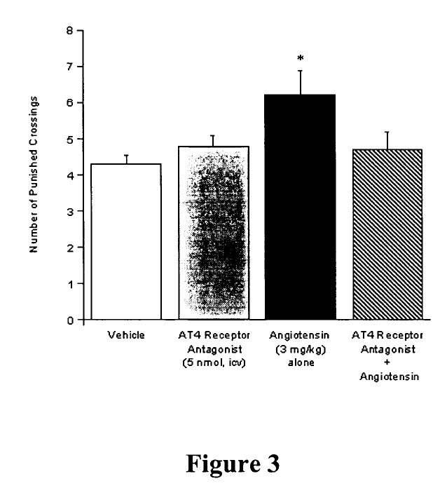

The results are shown in FIG. 3. Acute treatment with 5 nmol (icv) of the AT4 receptor antagonist, divalinal, had no effect on behavior when tested alone. However, divalinal completely blocked the anxiolytic-like effects of angiotensin IV in the 4-plate model. These data show that the AT4 receptor, in part, mediates the anxiolytic-like properties of Ang IV.

The present invention is not limited to the embodiments described and exemplified above, but is capable of variation and modification within the scope of the appended claims.

What is claimed:

1. A method for treating a neuropsychiatric disorder in a subject in need of such treatment comprising administering to the subject a composition comprising a pharmaceutically acceptable carrier and an angiotensin IV receptor (AT4R) antagonist in an amount effective to diminish the biological activity of the AT4R.

2. The method of claim 1, wherein the neuropsychiatric disorder is anxiety.

3. The method of claim 1, wherein the AT4R antagonist induces a conformational change in the AT4R.

4. The method of claim 1, wherein the AT4R antagonist inhibits the active site of the AT4R.

5. The method of claim 1, wherein the AT4R antagonist is angiotensin IV, Divalinal-Angiotensin IV, LVV-hemorphin 7, Nle-Ang IV, or Norleucinal Ang IV.

6. The method of claim 1, wherein the subject is a mammal.

7. The method of claim 1, wherein the subject is a human.

8. A method for treating a neuropsychiatric disorder in a subject in need of such treatment comprising modulating the expression of an AT4R in the subject.

9. The method of claim 8, wherein the neuropsychiatric disorder is anxiety.

10. The method of claim 8 wherein expression of the AT4R is diminished by utilizing an oligonucleotide molecule that is antisense to a nucleic acid encoding the AT4R.

11. The method of claim 10, wherein the molecule that is antisense to a nucleic acid encoding the AT4R is a siRNA.

12. The method of claim 8, wherein the subject is a mammal.

13. The method of claim 8, wherein the subject is a human.

14. A method for treating anxiety in a subject in need of such treatment comprising modulating the localization of the AT4R to the cell membrane.

15. A method for treating anxiety in a subject in need of such treatment comprising modulating the concentration of anxiolytic or anxiogenic neuropeptides in the subject.

16. The method of claim 15, wherein the concentration of an anxiolytic neuropeptide is increased.

17. The method of claim 16, wherein the anxiolytic neuropeptide is oxytocin.

18. The method of claim 15, wherein the concentration of an anxiogenic neuropeptide is decreased.

19. The method of claim 18, wherein the anxiogenic neuropeptide is vasopressin.

20. The method of claim 15, wherein the subject is a mammal.

21. The method of claim 15, wherein the subject is a human.

22. The method of claim 15, wherein the concentration of anxiolytic or anxiogenic neuropeptides is in synapses.

23. A method for identifying antagonists of the AT4R comprising contacting a test compound with the AT4R and determining a decrease in the biological activity of the AT4R in the presence of the test compound relative to the biological activity of the AT4R in the absence of the test compound.

24. The method of claim 23, wherein the AT4R is bound to a cell membrane or cell membrane fragment.

25. The method of claim 24, wherein the wherein the cell membrane or cell membrane fragment is from a mammalian cell.

26. The method of claim 25, wherein the mammalian cell is a kidney cell, cardiac cell, adrenal gland cell, or brain cell.

27. The method of claim 25, wherein the mammalian cell is a stable cell or stable cell line expressing the AT4R.

28. A compound identified by the method of claim 23.

29. A pharmaceutical composition comprising the compound of claim 28 and a pharmaceutically acceptable carrier.

30. A method for identifying compounds that reduce anxiety in a subject comprising administering a test compound to the subject and determining a decrease in the level of anxiety in the subject relative to the level of anxiety in the subject in the absence of the test compound.

31. A compound identified by the method of claim 30.

32. A pharmaceutical composition comprising the compound of claim 31 and a pharmaceutically acceptable carrier.

33. The method of claim 30, wherein the subject is a mammal.

34. The method of claim 30, wherein the subject is a human.

35. A method for identifying compounds that reduce anxiety in a subject comprising contacting a test compound with the AT4R and determining a decrease in the biological activity of the AT4R in the presence of the test compound relative to the biological activity of the AT4R in the absence of the test compound, and, administering the test compound to the subject and determining a decrease in the level of anxiety in the subject relative to the level of anxiety in the subject in the absence of the test compound.

36. The method of claim 35, wherein the subject is a mammal.

37. The method of claim 35, wherein the subject is a human.

38. A compound identified by the method of claim 35.

39. A pharmaceutical composition comprising the compound of claim 38 and a pharmaceutically acceptable carrier.

40. The method of claim 35, wherein the ATR4 is bound to a cell membrane or cell membrane fragment.

41. The method of claim 40, wherein the cell membrane or cell membrane fragment is from a mammalian cell.

42. The method of claim 41, wherein the mammalian cell is a kidney cell, cardiac cell, adrenal gland cell, or brain cell.

43. The method of claim 41, wherein the mammalian cell is a stable cell or stable cell line expressing the AT4R.


SEQUENCE LISTING (23 sequences)

SEQ ID NO: 1 - protein, 6 aa, Homo sapiens
VYIHPF

SEQ ID NO: 2 - protein, 10 aa, Ovis sp.
LVVYPWTQRF

SEQ ID NO: 3 - protein, 9 aa, Ovis sp.
LVVYPWTQR

SEQ ID NO: 4 - protein, 7 aa, Ovis sp.
VVYPWTQ

SEQ ID NO: 5 - protein, 5 aa, Ovis sp.
VVYPW

SEQ ID NO: 6 - protein, 5 aa, Ovis sp.
VVYPW

SEQ ID NO: 7 - protein, 4 aa, Ovis sp.
VVYP

SEQ ID NO: 8 - protein, 9 aa, Ovis sp.
VVYPWTQRF

SEQ ID NO: 9 - protein, 8 aa, Ovis sp.
VYPWTQRF

SEQ ID NO: 10 - protein, 7 aa, Ovis sp.
YPWTQRF

SEQ ID NO: 11 - protein, 7 aa, Ovis sp.
VYPWTQR

SEQ ID NO: 12 - protein, 6 aa, Ovis sp.
VYPWTQ

SEQ ID NO: 13 - protein, 5 aa, Ovis sp.
VYPWT

SEQ ID NO: 14 - protein, 4 aa, Ovis sp.
VYPW

SEQ ID NO: 15 - protein, 10 aa, Artificial Sequence
  Peptide analog of LVV-hemorphin-7
LVVAPWTQRF

SEQ ID NO: 16 - protein, 10 aa, Artificial Sequence
  Peptide analog of LVV-hemorphin-7
LVVYAWTQRF

SEQ ID NO: 17 - protein, 10 aa, Artificial Sequence
  Peptide analog of LVV-hemorphin-7
LVVYPATQRF

SEQ ID NO: 18 - protein, 10 aa, Artificial Sequence
  Peptide analog of LVV-hemorphin-7
LVVYPWAQRF

SEQ ID NO: 19 - protein, 8 aa, Ovis sp.
LVVYPWTQ

SEQ ID NO: 20 - protein, 7 aa, Ovis sp.
LVVYPWT

SEQ ID NO: 21 - protein, 6 aa, Ovis sp.
LVVYPW

SEQ ID NO: 22 - protein, 5 aa, Ovis sp.
LVVYP

SEQ ID NO: 23 - protein, 4 aa, Ovis sp.
LVVY